(19) United States
(12) Plant Patent Application Publication
(10) Pub. No.: US 2005/0241027 P1
(43) Pub. Date: Oct. 27, 2005

(54) Mango plant named "TFE 02"

(76) Inventor: Richard Elphick, Barbeton (ZA)

Correspondence Address: Catherine Anne Whealy;Proprietary Rights International, P.O. BOX 220, TOLAR, TX, 76476, US

(21) Appl. No.: 11/171,808

(22) Filed: Jun. 30, 2005

(30) Foreign Application Priority Data
Dec. 2, 2003 (ZA) PT 4043

(51) Int. Cl. A01H 5/00

(52) U.S. Cl. PLT/159

(57) ABSTRACT

A new and distinct cultivar of Mango plant named ‘TFE 02’, characterized by its upright and outwardly spreading tree form; vigorous growth habit; freely fruiting habit; time of fruit maturity, mid to late season; and red-colored fruit.

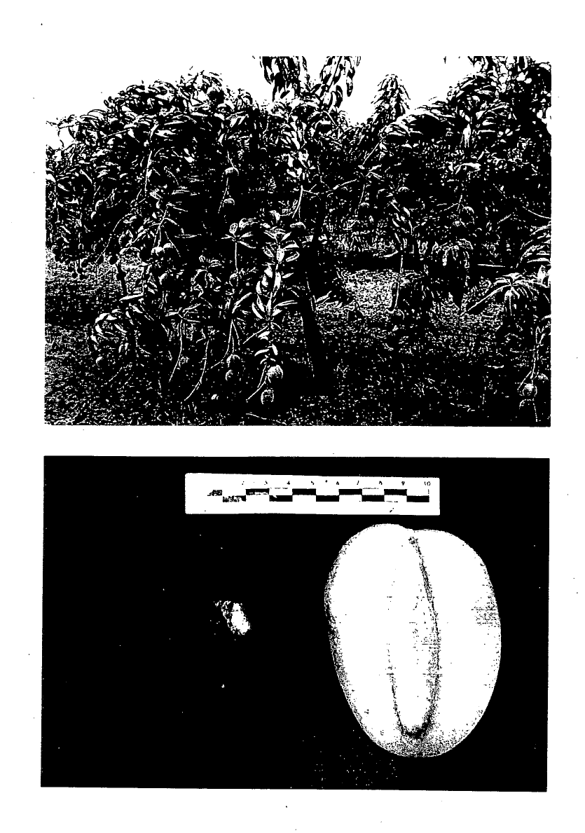

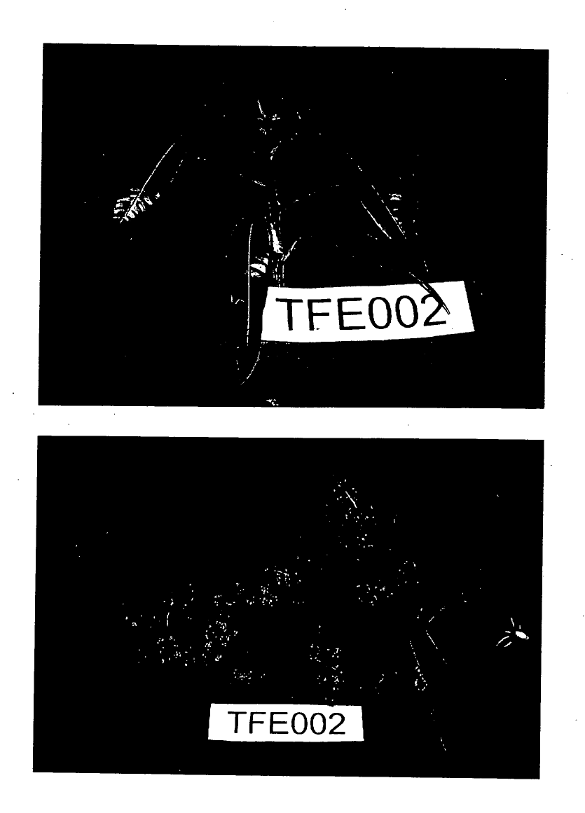

### BACKGROUND OF THE INVENTION

The present invention relates to a new and distinct cultivar of Mango plant, botanically known as Mangifera indica and referred to by the name ‘TFE 02’.

The new Mango is the product of a planned breeding program conducted by the Inventor in Malelane, Mpumalanga, Republic of South Africa. The objective of the breeding program is to create new Mango cultivars with precocious and reliable fruiting and medium-sized attractive fruit.

The new Mango originated from an open-pollination in Malelane, Mpumalanga, Republic of South Africa ofthe Mangifera indica cultivar Haden, as the female, or seed, parent with an unidentified selection of Mangifera indica. The new Mango was discovered and selected by the Inventor as a single plant within the progeny of the open-pollination in a controlled environment in Malelane, Mpumalanga, Republic of South Africa.

Asexual reproduction of the new Mango by grafting in a controlled environment in Hoedspruit, Republic of Republic of South Africa has shown that the unique features of this new Mango are stable and reproduced true to type in successive generations.

### BRIEF SUMMARY OF THE INVENTION

The cultivar TFE 02 has not been observed under all possible environmental conditions. The phenotype may vary somewhat with variations in environment such as temperature, daylength and light intensity, without, however, any variance in genotype.

The following traits have been repeatedly observed and are determined to be the unique characteristics of ‘TFE 02’. These characteristics in combination distinguish ‘TFE 02’ as a new and distinct cultivar:

1. Upright and outwardly spreading tree form.

2. Vigorous growth habit.

3. Freely fruiting habit.

4. Time of fruit maturity, mid to late season.

5. Red-colored fruit.

Plants of the new Mango can be compared to plants of the female parent, the cultivar Haden. In side-by-side comparisons conducted in Hoedspruit, Republic of South Africa, plants ofthe new Mango differed from plants of the cultivar Haden in the following characteristics:

1. Plants of the new Mango were more vigorous than plants of the cultivar Haden.

2. Plants of the new Mango had longer leaves than plants of the cultivar Haden.

3. Plants of the new Mango had shorter inflorescences than plants of the cultivar Haden.

4. Time of fruit maturity ofplants ofthe new Mango was later than time of fruit maturity of plants of the cultivar Haden.

5. Plants ofthe new Mango and the cultivar Haden differed in fruit skin and flesh coloration.

Plants of the new Mango can be compared to plants ofthe Mango cultivar Keitt, not patented. In side-by-side comparisons conducted in Hoedspruit, Republic of South Africa, plants ofthe new Mango differed primarily from plants of the cultivar Keitt in the following characteristics:

1. Plants of the new Mango were more vigorous than plants of the cultivar Keitt.

2. Plants of the new Mango had narrower leaves than plants of the cultivar Keitt.

3. Plants of the new Mango had shorter inflorescences than plants of the cultivar Keitt.

4. Time of fruit maturity of plants of the new Mango was earlier than time of fruit maturity of plants of the cultivar Keitt.

5. Plants of the new Mango and the cultivar Keitt differed in fruit skin and flesh coloration.

### BRIEF DESCRIPTION OF THE PHOTOGRAPHS

The accompanying colored photographs illustrate the overall appearance of the new Mango, showing the colors as true as it is reasonably possible to obtain in colored reproductions of this type. Colors in the photographs may differ slightly from the color values cited in the detailed botanical description which accurately describe the actual colors of the new Mango.

The photograph at the top of the first sheet comprises a side perspective view of a typical tree of ‘TFE 02’ with developing fruit. The photograph at the bottom of the first sheet is a close-up view of a typical fruit of ‘TFE 02’. The photograph at the top of the second sheet is a close-up view of typical leaves of ‘TFE 02’. The photograph at the bottom of the second sheet is a close-up view of a typical inflorescence of ‘TFE 02’.

### DETAILED BOTANICAL DESCRIPTION

In the following description, color references are made to The Royal Horticultural Society Colour Chart, 2001 Edition, except where general terms of ordinary dictionary significance are used. The aforementioned photographs and following observations and measurements describe plants grown in Hoedspruit, Republic of South Africa in an outdoor nursery and under conditions typical of commercial Mango production.

### Botanical Classification

Mangifera indica cultivar TFE 02.

### Parentage

Female, or seed, parent: Mangifera indica cultivar Haden.
Male, or pollen, parent: Unidentified selection of Mangifera indica, not patented.

### Plant Description

Appearance: Upright and outwardly spreading tree.
Growth rate: Rapid; vigorous.
Foliage description:
   Arrangement.—Alternate; simple.
   Length.—About 21.4 cm.
   Width.—About 5.25 cm.
   Shape.—Narrowly oblong.
   Apex.—Attenuate.
   Base.—Acute.
   Margin.—Entire.
   Aspect.—Twisting; concave.
   Texture, upper and lower surface.—Smooth, glabrous.
   Fragrance.—When developing leaves are crushed, terpinolene aroma is present.
   Petiole length.—About 6 cm.
   Color.—Developing foliage, upper and lower surfaces: 143A; towards the base, weak anthocyanin, N199B. Fully expanded foliage, upper and lower surfaces: 139A; venation, 144A. Petiole, upper and lower surfaces: 144A.

### Inflorescence Description

Appearance: Single flowers arranged in terminal panicles.
Inflorescence length: About 4.6 cm.
Flower bracts: Not observed.

### Fruit Description

Time of fruit maturity: Mid to late season.
Length: About 10.5 cm.
Diameter: About 8.7 cm.
Weight: About 376.33 grams.
Shape: Elliptic; neck not prominent; depth of stalk cavity shallow; groove in left shoulder shallow; sinus proximal of stylar scar absent; bulge proximal of stylar scar strongly expressed.
Texture, skin: Smooth, glabrous.
Texture, flesh: Fine, not fibrous.
Firmness, flesh: Firm.
Color, skin: 46B.
Color, flesh: 22A.
Turpentine flavor: Not detected.

### Disease/Pest Resistance

Resistance to known Mango pathogens and pests has not been observed on plants of the new Mango.

1. A new and distinct cultivar of Mango plant named ‘TFE 02’, as illustrated and described.
